(19) United States
(12) Patent Application Publication
(10) Pub. No.: US 2017/0332930 A1
(43) Pub. Date: Nov. 23, 2017

(54) SYSTEM AND METHOD FOR ECG SIGNAL PROCESSING

(71) Applicant: BTL HOLDINGS LIMITED, Nicosia (CY)

(72) Inventor: Martin Hanuliak, Praha 10 (CZ)

(21) Appl. No.: 15/159,104

(22) Filed: May 19, 2016

(51) Int. Cl. A61B 5/04; A61B 5/0408; A61B 5/0452

(52) U.S. Cl. CPC A61B 5/04012; A61B 5/0452; A61B 5/0408; A61B 2503/10

(57) ABSTRACT

Methods and systems provide for quick and precise analysis of ECG data, with a simple and understandable visualization and an effective way of communicating the proposed diagnosis suggestion to the medical personnel. Systems and methods detect electrical potentials from at least one lead and process at least one signal. The measurement itself may be executed on the raw signal, to compare measured parameters with a set of criterions related to various diseases, for example to sudden death syndrome.

### FIELD OF INVENTION

The present invention relates to a method and system for the automatic processing of signals representing the electrical activity of heart. Methods and systems provide for electrocardiography data interpretation in shorter time with improved measuring means and visualization of signal.

### BACKGROUND

The electrocardiography (“ECG”) measures the electrical activity of the heart using electrodes placed on a body. The activity is measured over a period of time and reflects the direction of electrical current generated by a depolarization and a repolarization of cardiac cells. Heart activity may be tracked by a conventional system of three, five, seven twelve or sixteen leads, although even different numbers might be used.

A conventional 12-lead ECG captures tracing of the electrical activity from 12 different views of the heart muscle. The 12-leads include three bipolar limb leads, three unipolar augmented limb leads and six unipolar chest leads. This type of ECG also records the mean instantaneous vector representing the force and direction of the wave of the depolarization and repolarization through the heart. The mean instantaneous vector is also called the electrical axis.

Besides the 12-lead conventional system, other systems with various numbers of electrodes may be used to monitor the electrical activity of the heart muscle.

The recorded ECG signal is represented by a number of waves generated by individual leads. The ideal wave refers to a series of beats, which are represented by waveforms, where the deflections are typically marked as PQRST. Sixth and subsequent components, e.g. a U waveform may be visible under certain conditions. These deflections show flow of electric impulses through the heart.

Waveform analysis, referred to as a measurement, provides a set of parameters for every beat which may point towards deviations in the function of the cardiac muscle. The ECG parameters determined by the measurement are frequently used to determine a diagnosis of the heart muscle.

The recorded ECG signal may be recorded on graph paper as an analog signal. Analysis of this signal is slow and makes precise measurement of parameters difficult. The ECG signal may be also visualized as a digitalized signal, where the plurality of beats may be averaged to provide an averaged beat. In fact, the averaging of the beats to provide an averaged beat is a standard procedure of signal digitalization. The analog report does not provide an averaged report.

The ECG signal recorded by the plurality of leads is usually averaged by spatial or temporal averaging to produce a beat representing an average shape of all beats. Both techniques are based on the assumption that noise is random, whereas the signal has repetitive characteristics. Spatial averaging originates from multiple inputs by use of multiple closely spaced electrode pairs placed on the body. These inputs are averaged to provide noise reduction. Spatial averaging is able to provide an averaged ECG from a single beat, allowing a real-time beat-to-beat analysis, but requires electrical shielding of the patient. A second methodology, namely temporal averaging, uses a large number of beats (typically 10 or more). The digitalized beats are aligned and averaged with a recognition template to reject noisy beats. As a result the averaged signal cancels noise, which can be described as a number of electrical impulses that do not occur as a repetitious pattern or with the same consistent timing as the representative beat. However, a problem of the averaging methods is that it leads to loss of part of the information and increases the possibility of a wrong diagnosis. In some cases, automated ECG fails substantially in comparison to the manual approach.

The recorded signal is often interpreted using an ECG paper report. Parameters, which are capable of providing a diagnostic suggestion of a cardiac disease, are measured by many manual and time-consuming techniques from every single waveform, single beat or plurality of beats originating from every individual lead. Although the manual method may be preferred among general medical personnel, satisfactory execution of the analysis requires skill. The prolonged time of analysis and required skills when using the traditional measuring methods, in a stressful medical practice, may result in a wrong diagnosis. Also, visualization of the recorded signals on simple graph paper does not allow exact comparison of beats by the naked human eye.

For example, important QRS deflections may have different shapes on every lead and they may not start from the same spot on the different leads. Although the ISO standard provides the types of the leads used for localization of the QRS deflections, it is difficult and time consuming to follow due to the need of knowledge of different standards of various patients and diseases. This may lead to non-uniformity of the ECG analysis executed by different hospital departments. In addition, the location of the QRS deflection is an important difference between young healthy people and adults and sick patients.

In another example, the T deflection of one beat may partially overlap the P deflection of the following beat, therefore the T deflection does not reach the baseline and both deflections are not separated. This example can be observed predominantly on the ECG signal of young populations, but it may also occur in others.

Measurement of the non-averaged ECG signal and/or evaluation of the parameters may point towards a number of heart diseases, including arrhythmias, suspected heart attacks, seizures, ischemia, hypertrophies, myocardial blocks, and others.

The ECG analysis may be also used to predict the possibility of sudden cardiac death. One of the proposed set of criterions is designed for athletic youth. The set of standardized criterions is defined on the parameters measured on the ECG waves derived from individual leads of the conventional 12-lead ECG system. As a result, averaged ECG cannot be used for the loss of information during the signal averaging. A similar set of criterions was proposed for a diagnosis of Brugada syndrome and other serious diseases.

In light of the above, it is desired to provide a method and system for quick measurement and analysis of ECG raw data in order to analyze a large set of criterions and visualization of the calculated parameters with visualized limits of the predetermined criterions to allow medical personnel to evaluate the level of parameter breaching

### SUMMARY OF INVENTION

Methods and systems provide for quick and precise analysis of ECG data, with a simple and understandable visualization and an effective way of communicating the proposed diagnosis suggestion to the qualified person. Systems and methods detect the electrical potentials from at least one lead and process at least one signal without any type of averaging. The measurement itself may be executed on the raw signal, to compare measured parameters with a set of criterions related to various diseases, for example to sudden death syndrome.

Heart activity may be tracked by a conventional system of three, five, seven, twelve or sixteen leads. The system is not limited to the particular number of the leads, but it may consist of any other number of leads. The signal is subjected to a measurement, where the parameters are calculated and compared to a predefined set of criterions. A proposed diagnosis suggestion is based on a predetermined set of criterions, which may aid the qualified person in a selection of the precise diagnosis. The qualified person may check, adjust and/or change the parameter measurement to assess or correct the level of at least one criterion breach and in order to influence the diagnosis proposal.

According to another embodiment the qualified person may add at least one new criterion to the already existing set of criterions to make a precise proposed diagnosis.

According to still another embodiment the qualified person may create a new set of criterions to adapt the system and method for providing a diagnosis suggestion to any other disease or population of patients.

According to still another embodiment, a method selects a typical beat from plurality of beats of the certain lead, plurality of beats of all leads; and/or a selection of beats from any lead performed by qualified person.

Visualization of the parameters and displayed criterions of the parameters along with the diagnosis suggestion helps the qualified person to support or correct the possible diagnosis. In case of typical beat selection, the typical beat may be superimposed by at least one beat originating from the same lead. The typical beat may be also superimposed by at least one beat from another lead. For comparison, signals of additional beats may possess a certain degree of transparency according to their similarity to the typical beat.

In another embodiment, the typical beat may be zoomed-in to provide more detailed information. Also, the mathematical basis of every calculated parameter may be visualized together with the related measuring instrument, which may contain colored parts to distinguish the severity of breach of at least one criterion from a set of criterions. The rulers may also show border lines demonstrating the measurement of normality/abnormality and therefore it shows the part of the ECG signal potentially not meeting the limits of criterions.

The invention also extends the utility of ECG to various parts of population. The independence of the present invention from any type of reference allows detection of possible heart diseases on different groups of people, e.g. athletic youth, sporting population and people living with high-altitude adaptation. The selection of the typical beat provides the analysis of the ECG signal of different types of people. Their ECG signal may be atypical by the influence of many factors, for example breathing. When the typical beat is selected from the plurality of the recorded beats, the influence of the environment or the patient is not manifested on the ECG analysis.

### GLOSSARY

The term “signal” means a curve representing electrical potential recorded by at least on ECG lead.

The term “parameter” means a measurable factor that describes at least one characteristic of at least one signal and/or wave entity (e.g. distance between wave deflections, amplitude or inclination).

The term “measurement” means a process of obtaining at least one parameter from at least one single beat or the entire ECG recorded signal.

The term “criterion” means at least one parameter restricted by limits. Usage of criterion results in diagnosis suggestion. Criterion may be predetermined by system and/or a qualified person.

The term “patient” means a human or animal.

The term “beat” means a signal wave's deflections PQRST or at least one deflection present on the ECG signal from at least one lead. The beat may be subjected to measurement.

The term “typical beat” means a beat or at least one of deflections selected from at least one lead by an algorithm and/or by a qualified person.

The term “caliper” means a position mark on the displayed ECG signal defining at least one region of the signal.

### BRIEF DESCRIPTION OF THE DRAWINGS

FIG. 1 is an illustration of an ECG recording that describes the various entities of a voltage signal of a normal heartbeat.

FIG. 2 is a block diagram illustrating a non-limiting example of a method.

FIG. 3 is an exemplary illustration of a visualization of the analyzed ECG data.

FIG. 4 is an exemplary illustration of a visualization of a typical beat superimposed on other beat.

FIG. 5 is an exemplary illustration of a visualization of the single beat with a measuring device.

All of the figures show examples and are not intended to be limits on the scope of the invention.

### DETAILED DESCRIPTION

The principles and execution of the method and the apparatus may be better understood with reference to the drawings and the accompanying description of the non-limiting, exemplary embodiments.

Electrocardiography is a technique of recording the electrical activity of the heart muscle using electrodes placed on the body, where electrodes define leads. Individual leads provide a view of the electrical activity between a positive and negative pole. Heart electrical activity may be recorded by a conventional system of three, five, seven, twelve or sixteen leads. The system and method described below is not limited to mentioned number of the electrodes and leads, but it may consist of any other number of ECG leads and/or electrodes. In another embodiment, not all the leads connected to the patient may be required to be used in order to monitor ECG signal. In still another example, the electrodes may be placed in a special position to measure a derived ECG.

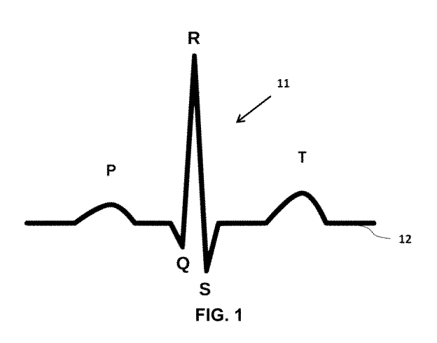

FIG. 1 shows a simplified representation of one beat 11 shown on ECG signal 12 recorded by an individual lead. The deflections forming the beat 11, markers PQRST, represent flow of the electric impulses through the heart muscle. Deflection U, which may follow the T deflection, is not shown on this figure as it may not appear on every ECG signal 12. It should be noted that the shape and direction of the deflections may be different according to the type of the lead used to the ECG signal recording.

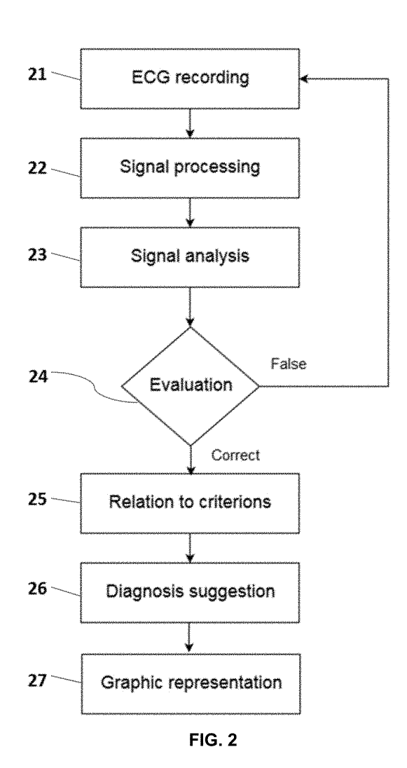

FIG. 2 illustrates exemplary block diagram of an ECG analysis process. Recorded ECG data may be subjected to signal processing and signal analysis. Analyzed data may be then related to a predetermined set of criterions and the proposal of a diagnosis suggestion is provided to the qualified person.

ECG data recording 21 may be executed by at least a pair of electrodes and/or at least one unipolar electrode connected to the patient. In another embodiment, the signal recording may be preferably executed by a standard 12-lead system. Signal recording may last from 0.5 seconds to 3600 seconds. In another embodiment, it may last from 1 second to 1800 seconds. In still another embodiment, it may last from 5 seconds to 60 seconds. In another embodiment not all the connected leads may be used to detect an ECG signal. The ECG data recording means storing the data for further processing and analysis. Recorded data may also be stored in volatile or non-volatile memory or transferred to storage unit of a Hospital Information System for later use.

Signal processing 22 may include at least one of filtering, multiplication, exponentiation, correlation, derivation, integration, and the like. Filtering may remove unwanted components of the signal such as baseline drift, motion artifacts, muscle artefacts, power line interference and/or EMG from the chest wall. Signal processing may be executed in different times during and after the ECG data recording 21. For example, it may be executed during the data recording. This exemplary embodiment is advantageous during a stress test of the patient, when the qualified person may judge the results of the method before the end of the procedure. In another embodiment, the signal processing may be executed immediately after the data recording. In still another embodiment, the signal processing may be executed after the data recording.

Signal analysis 23 may include at least one of detection of the beats, measurement of the beats and/or selection of the typical beat. Beats may be detected by various methods and algorithms. In one embodiment the beats may be detected by application of an adaptive algorithm searching for the maxima of the signal. In another embodiment the beats may be detected by the comparison of at least two leads and/or application of an adaptive algorithm searching for the global and/or local maxima of the signal. In still another embodiment, the beats may be identified by multiplication of the signal of an individual leads by itself and detection of the points with slope greater than a predefined slope threshold.

The beat detection may be followed by signal measurement, which may provide at least one parameter characterizing a detected ECG signal and/or detected deflections. The parameters may include P wave duration, PR interval duration, QT interval duration and any other parameter predefined by a system and/or qualified person. Signal measurement may be executed without any type of averaging process on a selection of beats described below.

The measurement of the parameters may be executed on at least one beat of at least one lead. In another embodiment, the measurement may be executed on at least one beat of all available leads. In still another embodiment, the parameters may be measured on all the beats on at least one lead. In another embodiment, the measurement may be executed on all identified beats of all available leads. In still another embodiment, the measurement may be executed on a set of identified beats of individual leads selected by qualified person. In another embodiment, the measurement may be executed on at least one beat selected by the qualified person.

The plurality of beats of at least one lead may be averaged by an averaging process and used for ECG analysis in an averaged manner. In order to provide visualization, averaged data may be subjected to measurement and the resulting averaged beat may be displayed to provide the qualified person with other information distinguished from the typical beat.

Selection of the typical beat may follow the measurement and may be accomplished by application of selection attributes. The typical beat may be selected as the beat reaching the best selection score in all available selection attributes. The typical beat may be selected as the beat reaching the best selection score in at least one selection attribute described below. Other beats may also be scored for the type of visualization described below. The selection attributes of the typical beat may include signal/noise ratio, QRS complex width, ST segment duration, ST interval duration, QT interval duration, PR segment duration, PR interval duration, PQ interval duration, presence of U deflection, amplitude height of QRS deflections, RR duration RR duration, RR interval stability, QRS axis and/or others. Preferred attributes used for selection of the typical beat may be at least one, more preferably at least two of these attributes. Even more preferred set of attributes may be signal/noise ratio, RR duration and at least one other mentioned selection attribute. Most preferred set of attributes may be signal/noise ratio, RR duration, amplitude height of QRS deflections and QRS complex width.

The system and method may select the typical beat by application of at least one selection attribute. The attribute or attributes may include a scale of importance applied during the selection. The scale of importance of any individual attributes may be changed by the qualified person.

Selection of the typical beat through the application of the attributes may be executed on the plurality of the beats of an individual lead by implemented software comparison to find the typical beat for all the leads. The typical beat is preferably a group of the same beats on various leads. Therefore, using e.g. 12 lead ECG and finding the typical beat, allows viewing the electrical vector of the typical beat from 12 different positions. In an alternative embodiment, the typical beat may be established for each lead separately. In another alternative embodiment, the qualified person may preselect the plurality of the beats from any available leads in order to create a base for selection of the typical beat.

When the typical beat cannot be selected due to low quality of the ECG signal, the system will not allow the continuation of the procedure and inform the qualified person to repeat ECG signal recording. For example, the low quality of the ECG signal may be characterized by high power interference noise from motion artifacts, muscle artifacts, power line interference and/or EMG from the chest wall.

It may be also executed on the plurality of the beats of the plurality of available leads. In another embodiment, the typical beat may be selected directly by a qualified person as a one particular beat.

In still another embodiment, the qualified person may select a one or more beats from any individual lead to create a basis for typical beat selection. This may allow the qualified person to focus on a beat and/or a typical beat of a plurality of beats with minor alterations which are not visible at other beats.

Calculated parameters of all beats may be then stored in a storage unit and compared to the at least one criterion in next step of the diagram.

In another embodiment the selection of typical beat is not required and the selected set of criterions may be compared with the measured parameters of any beat and/or the set of a beats selected by a qualified person from the all beats of all leads. It may allow the qualified person to focus on every single beat and/or the whole set of beats without a necessity to obtain a typical beat.

Detection of the beats, measurement of the beats, selection of the typical beat, and signal recording and signal processing, may be evaluated during the evaluation 24 in order to ensure the quality of the results. In another embodiment, the evaluation may be executed during the whole process of ECG analysis immediately after each event of the block in the block diagram. It may also signal the need for new measurement to the qualified person by any human perception form. Alternatively, the new measurement may immediately follow after the evaluation of an imperfection.

Parameters calculated during the signal analysis may be then subjected to relation to criterions 25, which may be predetermined to fit the characteristic of the possible alterations and/or diseases of the heart and/or another body system. Parameters are compared to the limits of at least one of the criterions and the subsequent results may be used to visualize the abnormality of the signal on the display. The method and system may provide a diagnosis suggestion 26, which may aid the qualified person with the diagnosis conclusion. According to still another embodiment, the method and system may provide a diagnosis conclusion.

As many parameters may be measured on every available beat, the system and/or the qualified person may select the typical beat to enable significant reduction of time. In the preferred embodiment the measured parameters may be compared with the plurality of criterions. In the preferred embodiment the system and method compares criterion or criterions on at least 3 leads of the ECG system, more preferably on at least 5 leads of the ECG system, most preferably on at least 12 leads of the ECG system and the total number of criterion comparisons is at least 3, more preferably at least 5, even more preferably at least 12 and most preferably at least 18. Criterions used to predict possible risk may be selected for example from the set of criterions related to the athletic youth, Brugada syndrome criterions or any other criterions used by medical professionals. In another embodiment, the qualified person may add a new set of proposed criterions to an already existing set or create a new set of criterions to obtain a new form of ECG data analysis that may be helpful in diagnosis of the another alteration and/or disease.

Systems and methods of the invention may also produce information about the presence of a pacemaker implanted in the body of the patient, wrongly positioned ECG electrodes and/or other technical irregularities of the ECG recording. This information may be provided during the execution of any block mentioned in the block diagram illustrated by FIG. 2.

In the preferred embodiment the typical beat may not be produced by any averaging process, but may be selected from the plurality of identified and measured beats. However, according to still another embodiment, the typical beat may include also an averaged beat. The automated selection of typical beat may be also changed to another selection of typical beat by a qualified person. In that case, all parameters related to the typical beat may be automatically recalculated to fit the newly selected beat and compared to the limits of the predetermined criterions. In still another embodiment, the qualified person may select a set of beats to create a basis for typical beat selection. In still another embodiment beats may be selected by a qualified person to create an amendment to already selected typical beat. Amended beats may be visualized in a superimposition to the typical beat.

Final results of the analysis and proposed diagnosis suggestion may be transferred to a Hospital Information System database where the ECG data and analysis may be further studied and/or corrected by the qualified personnel.

In the case the beat of interest may not be detected correctly or the typical beat may not be detected at all, the qualified person may use calipers to identify at least one basic parameter of the beat. The algorithm may execute detection of all other parameters of the newly designated beat. Therefore the measurement of the parameters may be executed repeatedly in independent manner from the block diagram illustrated on FIG. 2. In a preferred embodiment, the basic parameters may include the width of QRS complex, RR duration, ST segment width, length of ST interval, length of QT interval, length of PR segment, length of PR interval and length of PQ interval.

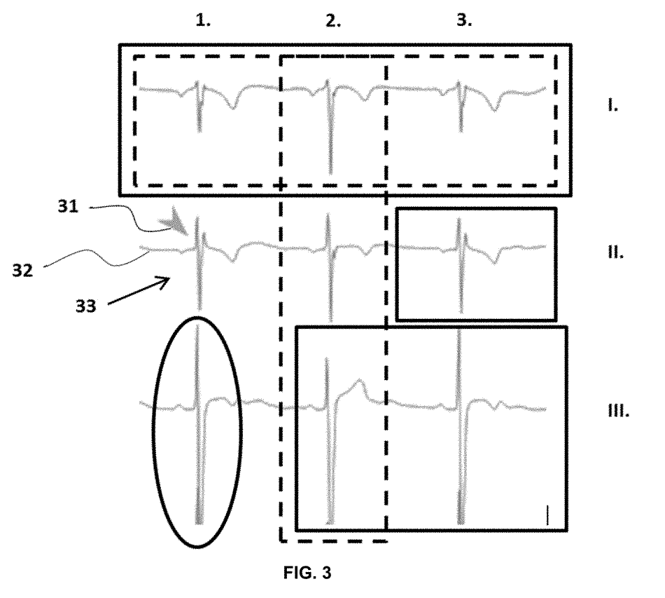

Referring to FIG. 3, the typical beat may be selected from:

1. The beats of one lead (in the horizontal direction along the time axis) as indicated by the horizontal rectangle shown in both solid and dotted lines in FIG. 3.

2. The plurality of beats of all available leads (in the vertical direction along the voltage axis) as indicated by the vertical rectangle shown in dotted lines in FIG. 3.

3. The plurality of beats of all available leads selected at random, as indicated by the rectangles in FIG. 3.

4. By the direct selection of the typical beat (e.g. by a qualified person) as indicated by the ellipse in FIG. 3.

These different forms of the typical beat may be selected from the same ECG signal. Therefore the individual ECG recording may provide different sets of typical beats.

After the diagnosis suggestion 26, the graphic representation 27 with all the related features of visualization described below may be shown.

FIG. 3 illustrates an exemplary visualization of three vertical groups of beats (1, 2 and 3) sequenced on the ECG signals from three leads. This type of visualization may display the ECG signal 32 with the representation of some measured parameters related to the beat 33 and/or plurality of beats and/or the signal itself. The displayed beats may be marked (e.g. by a symbol 31) to express critical, non-critical and/or frontier characteristics of their relation to a possible irregularity of their at least one measured parameter compared to a set of criterions. The displayed beats may be marked to express a critical and non-critical characteristic of at least one measured parameter. The visualization of different marks may distinguish between critical and/or noncritical breach or may distinguish between typical beat and beat.

The possible marking may include different coloring of every beat, symbol in the vicinity of the anomalous beat or particular part of the waveform and any other human perception form. The beat may be also marked by an arrow, as shown in FIG. 3. The marking 31 itself may draw attention of the qualified person to a problematic part of the ECG signal 32. In one embodiment the marked typical beat may be zoomed-in to visualize its anomalous attributes. In another embodiment the marked beat may be selected for detailed analysis without necessarily designating it as a typical beat, since not all of the criterion may be necessarily compared with the typical beat.

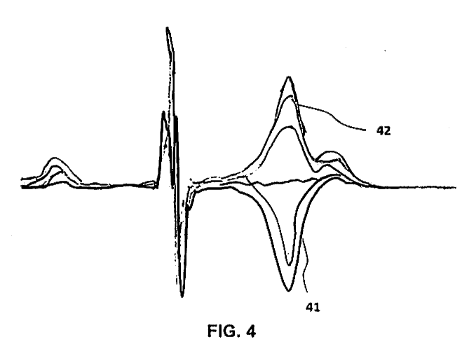

FIG. 4 illustrates an exemplary superimposition of the typical beat 41 on at least one other beat 42. According to the exemplary embodiment shown in FIG. 4, the typical beat 41 may be partially superimposed on at least one beat from the other lead or leads. However, the typical beat 41 may also be partially superimposed by at least one other beat from the same lead. In exemplary embodiment the typical beat 41 may be shown in solid line with no transparency superimposed by at least one beat 42 from at least one of the individual leads. In still another embodiment the typical beat may be superimposed by beats selected by random manner according to wishes of qualified person. The displayed other beats may be shown with a certain level of transparency distinguishing themselves from the typical beat displayed by solid line. The transparency of other beats may be based on plurality of the factors. The preferred set of factors is the selection score described above, a parameter similarity of other beats to the typical beat and/or a parameter similarity of the individual deflection of other beats to the typical beat. A more preferred set of factors is the selection score described above, the similarity of amplitude value of QRS deflection to the typical beat and the similarity of the QRS width to the typical beat. The most preferred factor is the selection score described above.

In one embodiment, the parts of superimposed beats with the exact overlay may be colored differently (e.g. different color, hue, luminance, saturation etc.) from the rest of the beats.

According to another example, when the typical beat is at any part superimposed by at least one beat, the almost exactly superimposed parts of both beats may be marked by different colors. The other part of the compared beat may be marked by red color to draw attention of qualified person to this particular part.

In another embodiment, the superimposition of the beats may be supplied with a text list of displayed beats. This list may serve to select another displayed beat other than typical beat. Selection of a different beat may be demonstrated by change of transparency. The newly selected beat may therefore be visualized as new solid beat. The typical beat may be therefore also displayed with certain degree of transparency, but without missing the typical beat status.

In still another embodiment, the anomaly in any visualized beat may be marked by an optional marking, which may include colored arrows, colored circles, colored parallel lines and other. The displayed marking may also share the degree of transparency according to marked beat.

This type of visualization may be advantageous for the qualified person because the superimposition of the signal provides information about other beats. As has been already noted, the non-averaged signal may include all beats with all their anomalies and provides the more broad information about the function of the heart. Using the described superimposition, the qualified person may focus attention on the typical beat visualized with no transparency. At the same time, the qualified person may also observe other beats possessing certain degrees of transparency. Such visualization may allow comparing the typical beat to another beat and noticing anomalies of other beats. In contrast to the standardized averaged beat analysis where the averaged beat is created from all beats, the typical beat may be one of recorded beats and still, it represents all other beats in the best possible manner. The visualization of the typical beat does not prevent the qualified person from deep analysis of other beats, which may lead to more precise diagnosis. This aspect is further strengthened by possible change of the transparency of the beats by a qualified person. Therefore the proposed superimposition provides advantage over analog reports printed on graph paper. The superimposed beats may be assessed and analyzed in significantly shorter time than the graph paper report.

It should be understood that the described features of visualization of beats may help the qualified person to compare all displayed leads, therefore the visualization may aid in selection of more precise diagnosis suggestion.

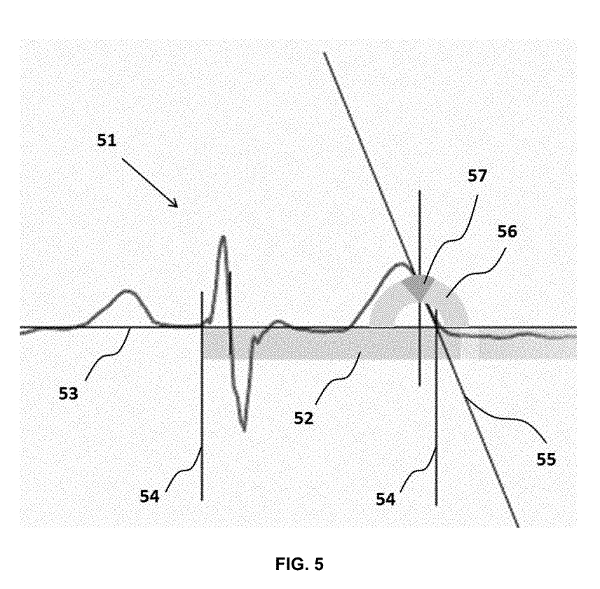

FIG. 5 illustrates an exemplary visualization of the beat 51 with a positionable measuring device 52, which may include a ruler, protractor, circular sector, French curve, tangent line, circle and the like. The measuring device may be able to visualize the measured parameter and/or the criterion in order to clearly inform the qualified person. The measuring device may comprise a measuring parameter scale and/or criterion scale. More preferably the measuring device may provide visualized information about various levels of criterion satisfaction. As an example, the measured non-critical parameters which are clearly out of the criterion's breach may be visualized by green color, the measured frontier parameters which are close to the criterion breach may be visualized by yellow color and the measured critical parameters which breach the criterion may be visualized by red color. The proposed system and method may be possible to display at least one measuring device. The measuring device may be displayed e.g. on horizontal axis, vertical axis, in angle or close to the measured waveform to the ECG signal. The beat may be a typical beat and/or zoomed-in individual beat selected by the qualified person.

In an exemplary embodiment the ruler 52 may be displayed in a horizontal manner under the baseline 53 to aid the qualified person with evaluation of the possible breach of the limits of the criterion testing the length of QT interval bordered by the calipers 54. Another exemplary shown element is the tangent 55 used to indicate the end of the T wave. The ruler may 52 be composed of a green element and a red element, where the green element marks the normal part of the tested parameter and the red element marks the breach of the limits of the criterion and an abnormal part of the parameter. In addition, the ruler may also include the yellow part marking the border lines of normality/abnormality, and therefore it may show the part of the ECG signal suspected of not meeting the limits of criterion. Another exemplary element shown is the tangent 55 used to indicate the end of the T wave. The semicircle 56 may include a circular sector 57 marking the border lines of normality/abnormality.

In another embodiment, the ruler may be displayed in a vertical manner. For example, the vertical ruler may aid with the evaluation of the possible breach of the limits of the criterion testing the depth of Q deflection. The displayed colored ruler may show the normal and abnormal depth of the tested parameter and aid the qualified person with a diagnosis suggestion concerning the pathologic shape and depth of the Q deflection.

In still another exemplary embodiment the French curve may be displayed in the vicinity of the ECG signal. It may aid the qualified person with determination of the unwanted curvature of the baseline. This may aid with the identification of the irregularities of amplitude height.

A displayed beat may be zoomed-in to display individual regions of the beat. It may be combined with already described embodiment concerning the visualization of the results.

The measuring devices may be displayed on visualization shown on FIG. 3. The measuring devices may be displayed independently for every beat of any lead.

Operation of the system may be improved based on corrective actions executed by the qualified person. The system may track the manual settings of the positions of the calipers, and correction of the measurement and/or the identification of the wave deflection. The system may also evaluate the selection of the typical beat and/or the selection of individual beat to improve the selection method. In addition, the system may boost evaluation by using data concerning the correction and/or denial of a suspected breach of the limits of at least one criterion.

The proposed system and method may be used for prediction of a suspected sudden death syndrome by comparing measured parameters to predefined sets of criterions. One particular group may be a set of criterions related to athletic and relatively healthy young population undergoing an intense training. Subsequent changes in heart physiology and instabilities of the ECG signal linked to respiratory rate observed in athletic young population are the main aspects prohibiting usage of averaged ECG to obtain diagnosis suggestion. The ECG waveforms may be influenced by breathing. During breathing the heart rate may vary. Mainly during the inspiration phase, the heart rate accelerates PR and QT intervals are reduced and amplitudes of QRS deflections are changed. On the other hand, during the expiration phase, the heart rate slows. Described anomalies may occur mainly in young populations and athletic populations. Therefore there is a need to provide a system and method for choosing a typical beat, which does not represent heart rate extremities.

Application of the criterions to predict the sudden death syndrome may include recording of the heart electrical activity by standard 12-lead ECG configuration. The individual beats are detected according to the above described method. In one embodiment the method may require identification of typical beat, which is selected from all recorded beats of the individual lead. It also means that the selection process may select a set of typical beats according to their respective leads.

The deflections of the ECG signal are subjected to signal measurement to provide a set of parameters. In one embodiment the measurement may be executed on all beats of all leads. In another embodiment the measurement may be executed on all beats of the selected at least one lead. In another embodiment the measurement may be executed on all leads of the at least one beat. In still another embodiment the measurement may be executed on at least one selected beat of at least one selected lead.

The comparison of the at least one parameter to at least one of the criterions results in the visualization of possible critical parts of ECG signal and/or diagnosis suggestion.

As an example, the parameter characterizing length of QRS complex duration may be compared to the criterion defining intraventricular conduction delay. When the time of duration exceeds for example 140 ms, the parameter breaches the limits of the criterion and the criterion is declared as positive. One positive criterion may be assessed as the positive result of the ECG screening and the patient may be under risk of the sudden death syndrome. The beat characterized by anomalous QRS complex duration may be visualized according to the preference of a qualified person.

The qualified person may not agree with the results of the ECG data analysis related to criterions. The qualified person may visualize the ECG signal together with the measuring device and criteria wording, then change the selected typical beat, correct the measured parameter (e.g. by calipers) and/or declare the related criterion as positive or negative. When the typical beat is changed to another beat, the parameters related to the ECG data may be compared to the criterions and new results may be visualized.

In another exemplary embodiment, the system and method may be used to aid in diagnosis of Brugada syndrome. Criterion described may encompass a proposed set of criterions used to diagnosis of this heart disease.

In another exemplary embodiment, the system and method be used to aid in prediction of the sudden infant death syndrome and others.

In another exemplary embodiment, the system and method be used to aid in prediction of the myocardial infarction and others.

All the methods, aspects, embodiments and examples described above may be combined together.

The foregoing description of a preferred embodiment of the invention has been presented for purposes of illustration and description. It is not intended to be exhaustive or to limit the invention to the precise form disclosed. Modifications and variations are possible in light of the above teachings or may be acquired from practice of the invention. The embodiments described explain the principles of the invention and its practical application to enable one skilled in the art to utilize the invention. Various modifications as are suited to a particular use are contemplated. It is intended that the scope of the invention be defined by the claims appended hereto and their equivalents.

1. A method for ECG analysis comprising:
recording electrical potentials from a patient by at least two electrodes creating at least one lead;
processing a signal from the at least two electrodes;
detecting beats;
measuring a plurality of parameters;
selecting a typical beat from non-averaged beats;
comparing at least one criterion with the plurality parameters of the at least one typical beat;
providing a notification of at least one criterion breach and/or suggesting a diagnosis.

2. The method of claim 1, where at least 3 leads ECG is used.

3. The method of claim 1, where at least 12 leads ECG is used.

4. The method of claim 1, wherein the processing a signal includes at least one of: filtration, multiplication, exponentiation, correlation, derivation and/or integration.

5. The method of claim 3, wherein the typical beat is selected from the plurality of the beats on 12 leads.

6. The method of claim 1, wherein the typical beat is selected from the plurality of the beats on each individual lead.

7. The method of claim 1, wherein the typical beat is selected from the plurality of the beats from any individual lead.

8. The method of claim 3, wherein the typical beat is chosen from at least one or more of the following selection attributes: signal/noise ratio, QRS complex width, amplitude height of QRS deflections and RR duration.

9. The method of claim 8, wherein the selection attributes possesses a scale of importance.

10. The method of claim 9, wherein the scale of importance is changeable.

11. The method of claim 8, wherein the at least one of the selection attributes is used to provide a selection score.

12. The method of claim 1, further including providing visualization of a breach of at least on criterion.

13. The method of claim 12, wherein the visualization contains a measuring device.

14. The method of claim 13, wherein the measuring device is positionable.

15. The method of claim 13, wherein the measuring device contains a measuring parameter scale.

16. The method of claim 14, wherein the measuring device contains a criterion scale,

17. The method of claim 13, wherein the measuring device provides visualised information on criterion satisfaction.

18. The method of claim 12, further including providing visualisation of marks.

19. The method of claim 1, further including providing visualisation of the beats by superimposition.

20. The method of claim 19, wherein superimposed beats are visualized with a degree of transparency.

21. The method of claim 20, wherein the degree of transparency is based on a selection score.

22. The method of claim 20, wherein the degree of transparency is based on at least one of selection score, similarity of amplitude value of QRS deflection to the typical beat and similarity of the QRS width to the typical beat.

23. A method of ECG analysis for detecting sudden death syndrome comprising:
recording electrical potentials from a patient by at least two electrodes creating at least one lead;
processing a signal from the at least two electrodes;
detecting beats of the heart of the patient;
measuring a plurality of parameters related to sudden death syndrome;
selecting a typical beat;
comparing at least one criterion with the plurality parameters of the at least one typical beat;
visualising a criteria breach with a measuring device containing a measuring parameter scale and a criterion scale providing visualised information about levels of criterion satisfaction;
providing notice of a criteria breach and/or suggesting a diagnosis.

24. The method of claim 23, further including providing visualisation of marks.

25. The method of claim 23, further including providing visualisation of the beats by superimposing beats, wherein the superimposed beats may be visualized with a degree of transparency.

26. A method of ECG analysis for diagnosing sudden death syndrome comprising:
recording electrical potentials from a patient by at least two electrodes creating at least one lead;
processing a signal from the at least two electrodes;
detecting beats of the heart of the patient;
measuring a plurality of parameters related to sudden death syndrome;
selecting a typical beat;
comparing at least one criterion with the plurality parameters of the at least one typical beat;
visualising of the beats by superimposing beats, wherein the superimposed beats are visualized with a degree of transparency;
providing notice in human perceptible form of a criteria breach and/or suggesting a diagnosis.

27. The method of claim 26, further including providing visualization of a breach of at least one criterion.

28. The method of claim 26, wherein the visualization contains a measuring device.

29. A method of ECG analysis for sudden death syndrome comprising:
recording electrical potentials from a patient by at least two electrodes creating at least one lead;
processing a signal from the at least two electrodes;
detecting beats of the heart of the patient;
measuring a plurality of parameters related to sudden death syndrome;
selecting a typical beat;
comparing at least one criterion with the plurality parameters of the at least one typical beat, wherein the set of criterions is related to athletic youth, sporting population and people living with high-attitude adaptation;
providing notification of a criteria breach and/or suggesting a diagnosis

30. The method of claim 1, further including providing visualization of the breach of at least on criterion.
